(19) United States
(12) Patent Application Publication
(10) Pub. No.: US 2015/0230812 A1
(43) Pub. Date: Aug. 20, 2015

(54) FORCEPS DEVICE

(71) Applicant: HI-LEX CORPORATION, Hyogo (JP)

(72) Inventors: Akihiro Ametani, Hyogo (JP); Akihiro Miyata, Hyogo (JP)

(21) Appl. No.: 14/430,747

(22) PCT Filed: Oct. 11, 2013
(86) PCT No.: PCT/JP2013/077697

§ 371 Date: Mar. 24, 2015

(30) Foreign Application Priority Data
Oct. 12, 2012 (JP) 2012-226552

(51) Int. Cl. A61B 17/28

(52) U.S. Cl. CPC A61B 17/282

(57) ABSTRACT

A forceps device includes a grasp portion, an operation portion equipped with a sliding portion and a rotation operation portion, an inner cable, an outer casing, a grip portion to be held when operating the operation portion, and a resin-coated portion. The proximal end side of the inner cable is mounted to the sliding portion and the distal end side is connected to the grasp portion. The proximal end side of the outer casing is mounted non-rotatably to the rotation operation portion and the distal end side is engaged rotatably with respect to the resin-coated portion and immovably in an axial direction with respect to the resin-coated portion. The sliding portion is mounted non-rotatably and slidably relative to the rotation operation portion. The rotation operation portion is rotatably connected to the grip portion and is provided at a proximal end side of the grip portion.

### TECHNICAL FIELD

The present invention relates to a forceps device, more specifically to a forceps device with good operability and good durability, as wear in a cable connecting portion is not likely to occur.

### BACKGROUND ART

Conventionally, a forceps device inserted into a human body through an endoscope has been used to grasp an affected area or the like in a body cavity. Such forceps device comprises a pair of forceps pieces which can be opened and closed for grasping the affected area. An operating force of an operation portion at a proximal side of the device which is operated by a doctor or the like is transmitted via an operation wire to the pair of forceps pieces to open and close the forceps pieces in the affected area distanced far from the operation portion and grasp the affected area. However, in the affected area inside the body cavity, there is a case where the opening and closing direction of the forceps pieces is not appropriate with respect to the position of the affected part. Therefore, a forceps device having a mechanism for rotating the forceps pieces has been used.

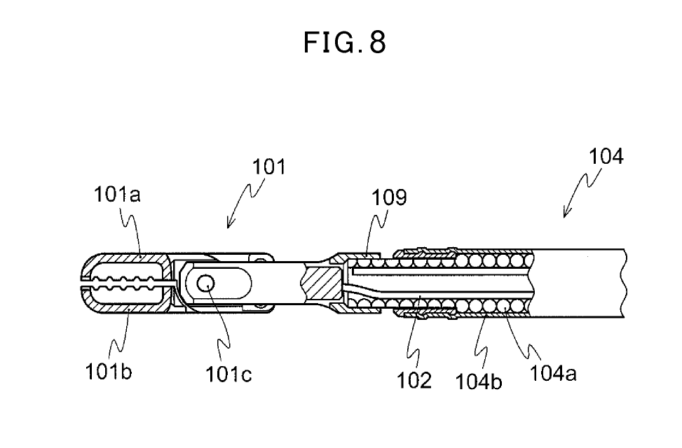

The endoscope treatment instrument disclosed in Patent Document 1 is known as such a mechanism for rotating forceps pieces. As shown in FIGS. 7 and 8, the endoscope treatment instrument 100 is equipped with a treatment portion 101 for performing a treatment on a tissue in a body cavity, an operation wire 102 connected to a proximal end of the treatment portion 101, an operation portion 103 for operating the treatment portion 101 that is connected to the operation wire 102, and an insertion portion 104 for connecting the treatment portion 101 and the operation portion 103. As for the treatment portion 101, as shown in FIG. 8, a pair of forceps members consisting of a 1st forceps member 101a and a 2nd forceps member 101b are rotatably connected to each other by a turning shaft 101c, while the operation wire 102 is connected at a proximal end side apart from the turning shaft 101c, and is connected to the operation portion 103 through the inside of the insertion portion 104. The insertion portion 104 comprises a coil sheath 104a, and an insulating tube 104b covering the periphery of the coil sheath 104a.

The operation portion 103 comprises an elongated main body 105, a rotational operation portion 106 attached to the main body 105 so as to be rotatable around the axis, and a slider 107 attached to the main body 105 so as to be slidable within a certain range in the direction of the axis. A handle 108 for finger hooking is provided at the proximal end of the main body 105. In addition, the rotational operation portion 106 consists of a tubular member 106a through which the proximal side of the insertion portion 104 is inserted, and a dial member 106b attached so as not to be rotatable with respect to the tubular member 106a.

In the endoscope treatment instrument 100 of the Patent Document 1 having such a configuration as mentioned above, when rotating the treatment portion 101, once the handle 108 (see FIG. 7) for finger hooking is held and the dial member 106b is rotated, the tubular member 106a being integrated with the dial member 106b and the coil sheath 104a being integrated with the tubular member 106a are rotated. As shown in FIG. 8, since the distal end side of the coil sheath 104a is integrated with the treatment portion 101 by means of a cover 109, the treatment portion 101 is rotated by the rotation of the coil sheath 104a. When the treatment portion 101 is rotated, the operation wire 102 is rotated by the treatment portion 101, and in order to release a rotational strain of the operation wire 102 that occurs at that time, the proximal end side of the operation wire 102 is rotatably connected to the slider 107 (a position indicated by reference numeral 107A in FIG. 7).

### PRIOR ART DOCUMENTS

### Patent Documents

Patent Document 1: JP 2010-42052 A

### SUMMARY OF THE INVENTION

### Problem to be Solved by the Invention

In the endoscope treatment instrument 100 of the Patent Document 1, the treatment portion 101 having the first and second forceps members 101a and 101b is rotated so as to be adapted to the orientation of the affected area by the construction described above. However, in the endoscope treatment instrument 100 of the Patent Document 1, the dial member 106b rotating the treatment portion 101 lies closer to the body of the patient than the handle 108 supporting the entire endoscope treatment instrument 100 when rotating the dial member 106b. Therefore, during the rotation of the dial member 106b, swinging is likely to occur on the axis of the proximal end side of the endoscope treatment instrument 100 (at the position where the handle 108 is provided). Furthermore, when sliding the slider 107 in order to open and close the first and second forceps members 101a and 101b of the treatment portion 101, it is necessary to re-hold the endoscope treatment tool 100 to prevent the swinging of the entire endoscope treatment instrument 100. In addition, when sliding the slider 107, if the sliding operation is carried out while holding the rotational operation portion 106, there is a possibility that the treatment portion 101, the position of which has already been adjusted, will rotate. Therefore, it is not possible to carry out the sliding operation while holding the rotational operation portion 106 and prevent swinging of the endoscope treatment instrument 100.

In addition, in the endoscope treatment instrument 100 of the Patent Document 1, the proximal end side of the operation wire 102 is rotatably connected to the slider 107. Without using a member for preventing wear of the operation wire 102 at the connection portion with the slider 107, the operation wire 102 would be worn out by its relative rotation with the slider 107, and thus there is a problem that the number of parts will be increased in order to prevent wearing.

Therefore, in the light of the mentioned problems, an object of the present invention is to provide a forceps device having good operability when operating the forceps device, and having good durability such that wear of the cable connecting portion is not likely to occur.

### Means to Solve the Problem

The forceps device of the present invention is a forceps device comprising: a grasp portion equipped with forceps pieces for grasping a tissue in a body cavity, being able to be opened and closed; an operation portion equipped with a sliding portion for opening and closing the forceps pieces of the grasp portion and a rotation operation portion for rotating the grasp portion; an inner cable for transmitting an operating force from the operation portion to the grasp portion, being provided between the grasp portion and the operation portion; an outer casing surrounding the inner cable; a grip portion to be grasped when operating the operation portion; and a tubular resin-coated portion for housing the outer casing rotatably, extending toward the grasp portion side from the grip portion, wherein a proximal end side of the inner cable is mounted to the sliding portion so as not to be rotatable, and a distal end side of the inner cable is connected to the grasp portion, a proximal end side of the outer casing is mounted non-rotatably to the rotation operation portion, and a distal end side of the outer casing is engaged rotatably with respect to the resin-coated portion and immovably in an axial direction with respect to the resin-coated portion, the sliding portion is mounted non-rotatably and slidably to the rotation operation portion, the forceps pieces of the grasp portion are opened and closed by sliding the sliding portion, the inner cable and the outer casing are rotated around the axis by rotating the rotation operation portion to rotate the grasp portion, and the rotation operation portion is rotatably connected to the grip portion, and is provided at a proximal end side of the grip portion.

In addition, it is preferable that a high frequency power supply terminal is connected to the inner cable or the outer casing, and the inner cable or the outer casing has an electric conductivity for enabling to supply high frequency current to the grasp portion from the high frequency power source terminal.

In addition, it is preferable that the high frequency power source terminal is penetrated through the grasp portion to be connected to the outer casing.

In addition, it is preferable that the high frequency power source terminal is provided at the rotation operation portion side, and is connected to the inner cable or the outer casing.

In addition, it is preferable that the rotation operation portion comprises a guiding portion for guiding the sliding portion, and a rotating portion provided closer to the proximal end side than the guiding portion and operated to perform the rotation operation.

### EFFECTS OF THE INVENTION

According to the present invention, it is possible to provide a forceps device having a good operability when doctors or the like operate the forceps device, and having a good durability such that wear on a connecting portion of an inner cable provided in the forceps device is not likely to occur.

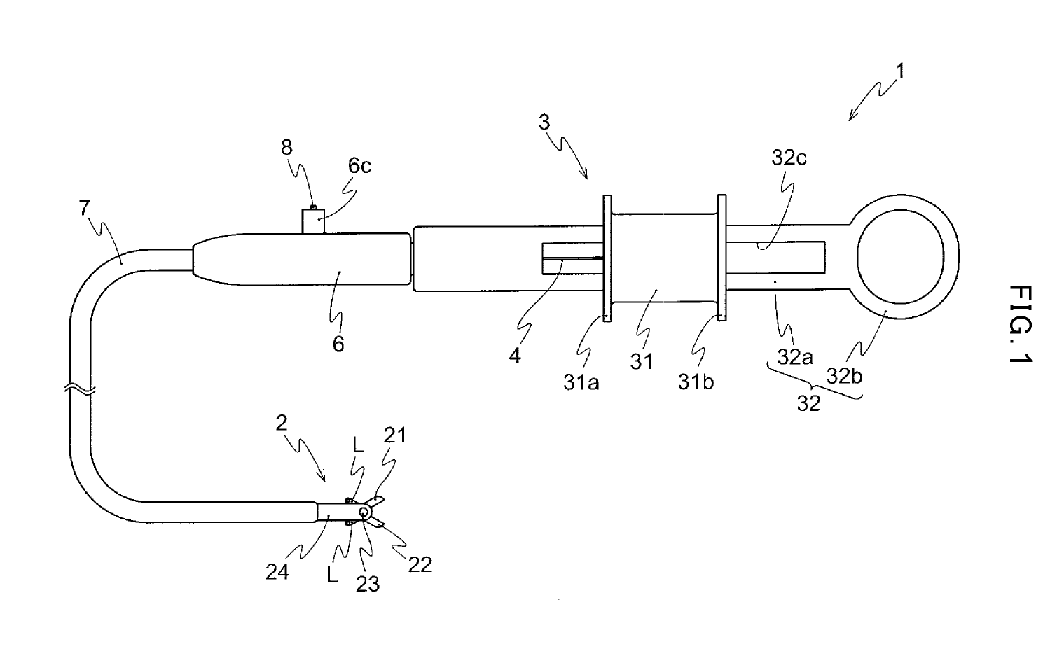

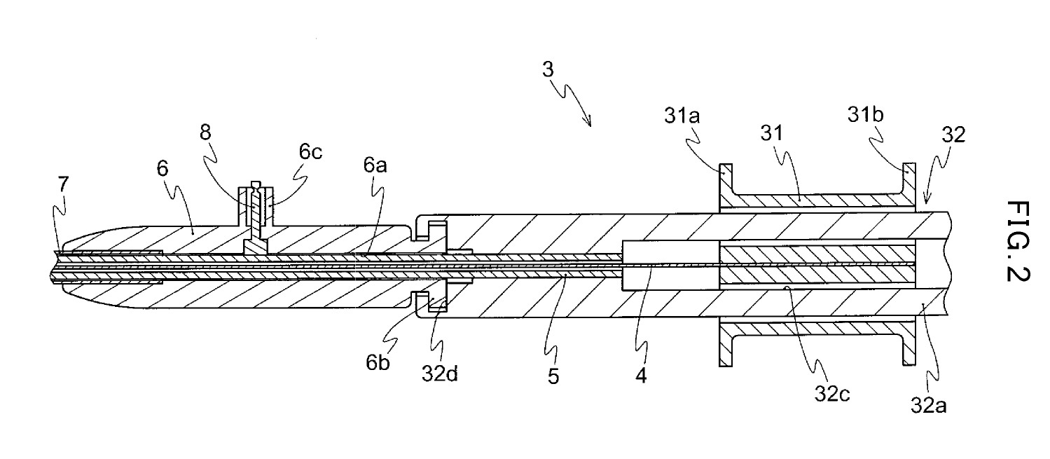

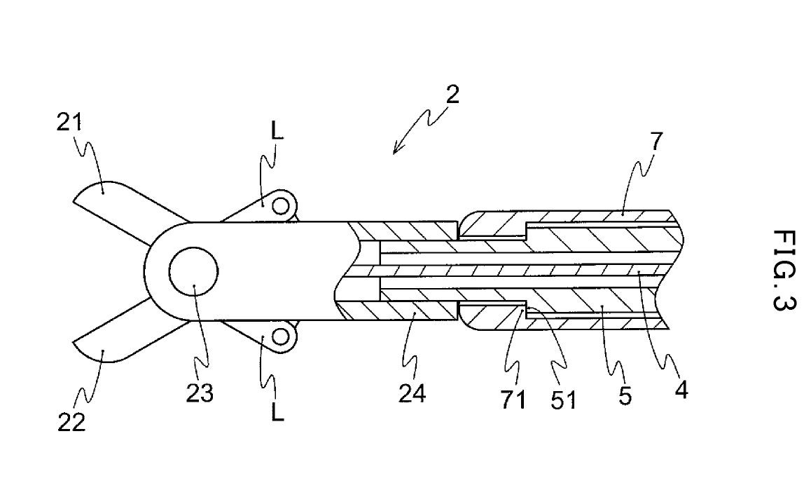

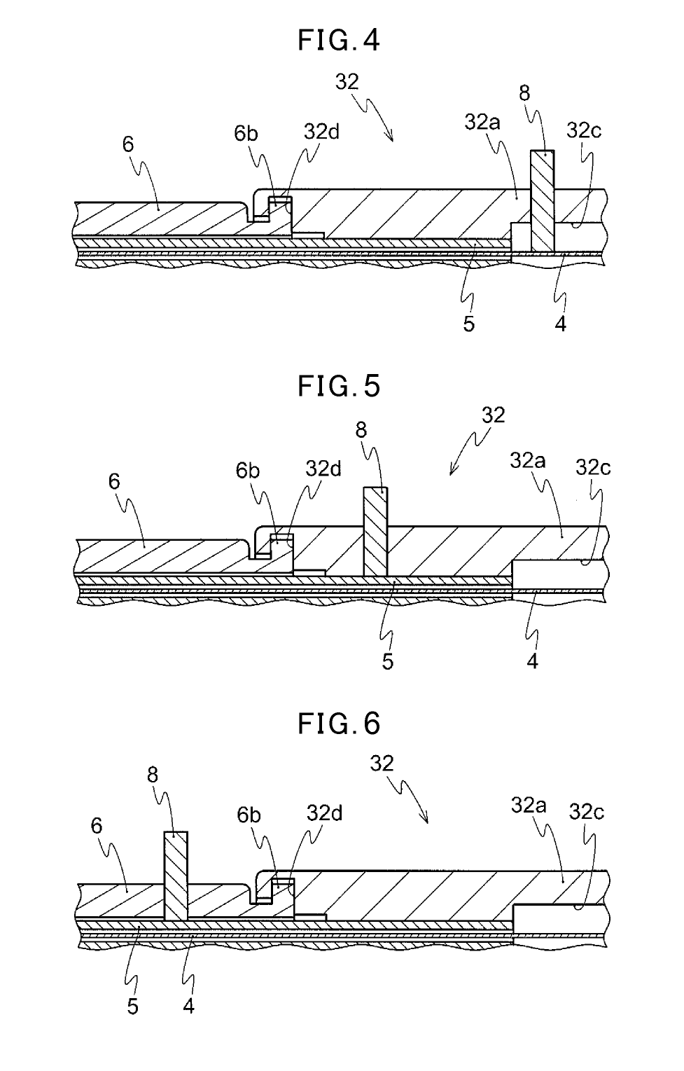

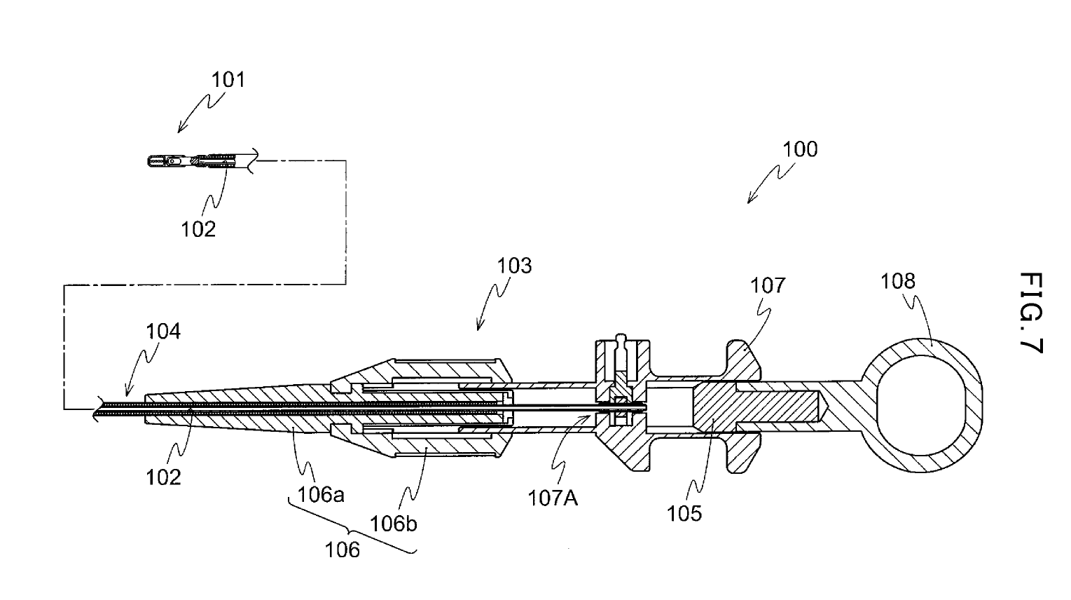

### BRIEF DESCRIPTION OF THE DRAWINGS

[FIG. 1] is an overall view of the forceps device of the present invention.

[FIG. 2] is a partially enlarged cross-sectional view showing the operation portion and the grip portion in the forceps device of the present invention.

[FIG. 3] is a partially enlarged cross-sectional view showing a connection state of the grasp portion in the forceps device of the present invention.

[FIG. 4] is a schematic diagram for explaining the mounting position of the high frequency power source terminal used on the forceps device of the present invention.

[FIG. 5] is a schematic diagram for explaining the mounting position of the high frequency power source terminal used on the forceps device of the present invention.

[FIG. 6] is a schematic diagram for explaining the mounting position of the high frequency power source terminal used on the forceps device of the present invention.

[FIG. 7] is an overall view of a conventional forceps device.

[FIG. 8] is an enlarged cross-sectional view showing a treatment portion of a conventional forceps device.

### EMBODIMENT FOR CARRYING OUT THE INVENTION

Hereinafter the forceps device of the present invention is described in detail by referring to the accompanying drawings.

As shown in FIG. 1, a forceps device 1 of the present invention comprises a grasp portion 2 having forceps pieces 21, 22, which can be opened or closed for grasping a tissue in a body cavity, and an operation portion 3 provided with a sliding portion 31 for opening and closing operation of the forceps pieces 21, 22 of the grasp portion 2 and a rotation operation portion 32 for rotating the grasp portion 2. Between the grasp portion 2 and the operation portion 3, an inner cable 4 is provided for transmitting an operating force from the operation portion 3 to the grasp portion 2. Around the inner cable 4, as shown in FIGS. 2 and 3, an outer casing 5 surrounding the inner cable 4 is provided, and guides the inner cable 4 from the operation portion 3 to the grasp portion 2. In addition, at the distal end side of the operation portion 3 there are provided a grip portion 6 for the practitioner such as a doctor to hold when operating the operation portion 3 and a tubular resin-coated portion 7 extending from the grip portion 6 towards the grasp portion 2 side and houses the outer casing 5 rotatably. It should be noted, herein, “distal end” in the component members refers to the side far from a practitioner such as a doctor who operates the forceps device 1 (i.e. a free end side of the grasp portion 2), and “proximal end” refers to the side closer to the practitioner such as a doctor who operates the forceps device 1 (i.e. a free end side of the rotation operation portion 32).

Forceps device 1 of the present invention is inserted into an endoscope (not shown) and introduced into a body cavity, and grasps tissues within the body cavity such as an affected area by remotely operating the forceps pieces 21, 22, and for example, performs cauterization and hemostasis of the tissues within body cavity by sending a high frequency current to the forceps pieces 21, 22.

Though the detail will be described below, during such operations, if the directions of the forceps pieces 21 and 22 of the grasp portion 2 are not appropriate relative to the orientation of the affected area, once the operation portion 3 is rotated, the grasp portion 2 rotates, which makes it possible to easily adjust the grasp portion 2 to face in a proper direction and perform the treatment in accordance with a location and orientation of the affected area.

As shown in FIGS. 1 and 3, the grasp portion 2 provided on the distal end side of the forceps device 1 is a portion for grasping the body cavity tissues, and a pair of forceps pieces 21, 22 is configured to be rotatable around a rotation axis 23 provided on a housing 24 of the grasp portion 2, and the forceps pieces 21, 22 open and close in response to the operation of the operation portion 3. As shown in FIGS. 1 and 3, the forceps pieces 21, 22 are configured such that a pulling operation of the inner cable 4 is converted to the opening and closing actions by a link mechanism L to open and close the forceps pieces 21, 22. Such a link mechanism L will not be explained in detail because well-known ones can be used and adopted appropriately according to an application and a desired operating action. It should be noted that, in order to open and close the forceps pieces 21, 22, it is not necessary to use the link mechanism L, and another mechanism may be used if it can convert the operating force of the inner cable 4 to the opening and closing actions of the forceps pieces 21, 22. In addition, while in the embodiments shown in FIGS. 1 and 3, the forceps pieces 21, 22 are configured to be operated by a single inner cable 4, they may be configured to be operated by two inner cables.

As shown in FIG. 1, operation portion 3 for operating the grasp portion 2 comprises a rotation operation portion 32 rotated by the practitioner such as a doctor, and a sliding portion 31 for operating the inner cable 4. The rotation operation portion 32 is a portion for applying a rotational operation when the practitioner rotates the grasp portion 2. The rotation operation portion 32 shown in FIG. 1 comprises an elongated substantially cylindrical body 32a, a guiding portion 32c for guiding the sliding portion 31, and a rotating portion 32b provided closer to the proximal end side than the guiding portion 32c and operated to perform a rotation operation. In FIG. 1, the guiding portion 32c is shown as a perforated slit having a length in the axial direction of the body 32a and provided on the body 32a of the rotation operation portion. The rotating portion 32b is shown as a ring-shaped member, but the shape is not limited. With respect to the sliding portion 31, the sliding portion is mounted non-rotatably and slidably to the rotation operation portion 32, and there is adopted, for example, an embodiment such that its convex portion provided inside thereof fits slidably to the guiding portion. The sliding portion 31 has a bobbin shape having flange portions 31a, 31b so as to be easily operated, and is configured so as to reciprocate between the distal end side and the proximal end side of the guiding portion 32c by the practitioner's operation.

As long as the rotation operation portion 32 and the sliding portion 31 have the shapes allowing a practitioner to operate them, the shapes thereof are not limited to the shapes shown in FIG. 1, and various shapes may be used. In addition, the portion which the practitioner will hold to perform the rotation operation (rotating portion 32b) may be provided on the position of the rotating portion 32b shown as a ring-shaped member in FIG. 1, or may be provided on the position of the body 32a of the rotation operation portion (i.e., if the rotation operation portion is rotated with the body 32a being held, the rotating portion may be the position of the reference numeral 32a in FIG. 1).

In addition, as shown in FIGS. 1 and 2, the forceps device 1 of the present invention has a grip portion 6, and the rotation operation portion 32 is rotatably connected to the grip portion 6, and is provided on the proximal end side of the grip portion 6. The grip portion 6 is a portion to be grasped when the practitioner operates the rotation operation portion 32. The grip portion 6 has an insertion hole 6a for inserting the inner cable 4 and the outer casing 5 therein, and is configured so as to allow the outer casing 5 housing the inner cable 4 to be rotated in the insertion hole 6a. The rotation operation portion 32 and the grip portion 6, as shown in FIG. 2, are connected and engaged with each other so that the rotation operation portion 32 does not move in the axial direction with respect to the grip portion 6. Such engagement makes rotatable connection possible by, for example, forming a disk-shaped cavity 32d in the body 32a of the rotation operation portion 32, and engaging the disc-shaped protrusion 6b of the grip portion 6 with the disc-shaped cavity 32d. It should be noted that the connection between the rotation operation portion 32 and the grip portion 6 is not limited to the structure shown in FIG. 2, and as far as the rotation operation portion 32 is able to rotate relative to the grip portion 6, other structures can be used.

In addition, in the embodiments shown in FIGS. 1 and 2, the high frequency power source terminal 8 is provided on the grip portion 6. The high frequency power source terminal 8 is provided in the insertion port 6c provided on the grip portion 6. The high frequency power source terminal 8 is provided on the grip portion 6 in FIGS. 1 and 2, but as described below, may be provided on the rotation operation portion 32. The high frequency power source terminal 8 is connected to the inner cable 4 or the outer casing 5, and since the inner cable 4 or the outer casing 5 has electrical conductivity which enables to supply a high frequency current to be sent to the grasp portion 2 from the high frequency power source terminal 8, it is possible to send the high frequency current to the forceps pieces 21, 22 of the grasp portion 2 and carry out cauterization of the affected part grasped by the forceps pieces 21, 22. The high frequency power source terminal 8 is connected via a cord to a high frequency power source which is not shown here. Since a configuration of the inner cable 4 or the outer casing 5 having electric conductivity is well-known, a description of its structure is omitted herein.

Because the high frequency current flows to the inner cable 4 or the outer casing 5, as shown in FIG. 2, the insulating resin-coated portion 7 is mounted on the distal end side of the grip portion 6 so that the high frequency current is prevented from flowing to the tissues other than the affected area in a body cavity. The resin-coated portion 7 has flexibility, and is configured to be able to move within the body cavity smoothly.

Next, a connection relationship of the inner cable 4 and the outer casing 5 with other members will be explained. As shown in FIGS. 2 and 3, the proximal end side of the inner cable 4 is attached to the sliding portion 31 so as not to be rotatable, and the distal end side thereof is coupled to the grasp portion 2. Thus, the inner cable 4 is operated by sliding the sliding portion 31, thereby opening and closing the forceps pieces 21, 22 of the grasp portion 2. Specifically, the inner cable 4 is mounted integrally with the sliding portion 31 by bonding or other known binding means, and is configured so as not to rotate around the axis with respect to the sliding portion 31. Thus, when the rotation operation portion 32 is rotated relative to grip portion 6, and as a result, the sliding portion 31 is rotated, relative rotation does not occur between the sliding portion 31 and the inner cable 4. Therefore, at the time of rotation operation, friction does not arise at the connection part between the sliding portion 31 and the inner cable 4, so wearing out of the inner cable 4 will not occur. The distal end side of the inner cable 4, as shown in FIG. 3, is connected to the forceps pieces 21, 22 via the link mechanism L, and the forceps pieces 21 and 22 are opened and closed by operating the sliding portion 31.

In addition, the proximal end side of the outer casing 5 is attached not to be rotatable with respect to the rotation operation portion 32, and its distal end side is engaged with the resin-coated portion 7 rotatably and immovably with respect to the resin-coated portion 7 in the axial direction. The inner cable 4 is slidably housed in the outer casing 5, and even in the body cavity where there is a curved portion, it is possible to transmit the operating force of the inner cable 4 to the grasp portion 2 side. The outer casing 5 is mounted integrally to the rotation operation portion 32 by bonding or other known binding means, and is configured so as not to rotate around an axis relative to the rotation operation portion 32. The outer casing 5 is not fixed to the grip portion 6, and is configured to be rotatable in the grip portion 6. Further, as shown in FIG. 3, an engaging portion 71 of the resin-coated portion 7 provided on its distal end side and projecting inwardly is engaged with the stepped portion 51 provided at the distal end side of the outer casing 5. The engagement between the outer casing 5 and the resin-coated portion 7 may be of any structure as long as the outer casing 5 can rotate in the resin-coated portion 7, and is immovable in the axial direction with respect to the resin-coated portion 7. The tip portion of the outer casing 5, as shown in FIG. 3, is fixed to the housing 24 of the grip portion 2. The housing 24 of the grip portion and the resin-coated portion 7 are configured to be rotatable to each other.

With the construction described above, by rotating the rotation operation portion 32, the inner cable 4 and the outer casing 5 rotate around the axis. And then, once the inner cable 4 and the outer casing 5 are rotated around the axis, the grasp portion 2 is rotated, thereby making it possible to adjust the direction of the forceps pieces 21, 22 in the body cavity. By the rotation of the rotation operation portion 32, the inner cable 4 and the outer casing 5 are rotated together, and therefore, a relative rotation does not occur between the inner cable 4 and the outer casing 5. Therefore, a frictional resistance in the rotating direction which occurs between the inner cable 4 and the outer casing 5 is not applied, and since the rotations of the inner cable 4 and the outer casing 5 are synchronized, the grasp portion 2 can be operated accurately.

Next, the position where the high frequency power source terminal 8 is provided will be described by referring to the schematic diagrams shown in FIGS. 4 to 6. In FIG. 4, the high frequency power source terminal 8 is provided on the rotation operation portion 32 side, and the high frequency power source terminal 8 is connected to the inner cable 4. In the embodiment shown in FIG. 4, since the inner cable 4 moves in the axial direction with respect to the high frequency power source terminal 8 by the operation of the sliding portion 31, for example, a high frequency power source terminal 8 of contact type such as sliding contact can be used. In FIG. 5, the high frequency power source terminal 8 is provided on the rotation operation portion 32 side, and the high frequency power source terminal 8 is connected to the outer casing 5. In the embodiment shown in FIG. 5, since the relative movement in the axial direction and circumferential direction do not occur between the high frequency power source terminal 8 and the outer casing 5, there is no wear caused on the high frequency power source terminal 8 and the outer casing 5. In FIG. 6, the high frequency power source terminal 8 penetrates the grip portion 6 and is connected to the outer casing 5. In the embodiment shown in FIG. 6, even when rotating the rotation operation portion 32 is rotated in order to rotate the grasp portion 2, the grip portion 6 does not rotate, and therefore, the high frequency power source terminal 8 does not rotate around the axis. Therefore, the cord for connecting the high frequency power source terminal 8 to the high frequency power source is not entangled by the rotation or the like, so that it is easy for the practitioner to operate. Moreover, the position of the high frequency power source terminal 8 does not change from its initial position when the forceps device 1 is inserted to an endoscope (the position in the direction of rotation is unchanged, and the high frequency power source terminal 8 facing upward in the initial position will not rotate and will not be facing downward at the stage of connecting the cord).

Therefore, when the practitioner connects the cord, the high frequency power source terminal 8 can be always kept in the position where the operation is easy. In addition, since the outer casing 5 rotates relative to the high frequency power source terminal 8, a mechanism such as a high frequency power source terminal 8 of contact type which maintains electric conductivity when relative rotation is carried out can be used.

Next, the operation of the forceps device 1 of the present invention will be described.

First, the practitioner operates the sliding portion 31 to bring the forceps pieces 21, 22 of the grasp portion 2 to the closed state, inserts the forceps device into the endoscope which is not shown here, and moves the grasp portion 2 to a position in a body cavity where there is an affected area.

When the grasp portion 2 is moved to the position of the affected area, the grip portion 6 is grasped and the rotation operation portion 32 is operated to change the direction of the grasp portion 2 while confirming with the endoscope. The grip portion 6 is in the position closer to the patient's body, and the rotation operation portion 32 to which a force is applied is closer to the practitioner side than the grip portion 6. Here, in the case where the rotation operation portion 32 and the grasp portion 6 are in a positional relationship opposite to the present invention, in other words, when the grip portion 6 is located on the proximal end side (on the hand side near the practitioner), and the rotation operation portion 32 is on the distal end side (on the side closer to the body of the patient), during the rotation operation, swinging with the hand side of the practitioner as an axis is caused at the distal end side by the operation of the rotation operation section 32, and the swinging is thus transmitted to the body of the patient and the endoscope. However, according to the structure of the present invention, the grip portion 6 on the side closer to the body of the patient is firmly held, and even if a force is applied to the rotation operation portion 32, and the swinging with the grip portion 6 as an axis occurs at the portion of the rotation operation portion 32, the swinging is suppressed by the grip portion 6 which is grasped at the side closer to the body of the patient than the rotation operation portion 32. Therefore, the swinging is not transmitted to the body of the patient and the endoscope, in which the forceps device 1 was inserted, and there is no burden on the patient.

The rotation of the grasp portion 2 is carried out, for example, by rotating the rotating portion 32b of the rotation operation portion 32. When the rotation operation portion 32 is rotated, the sliding portion 31 attached so as not to be rotatable to the rotation operation section 32 is also rotated. When the rotation operation portion 32 and the sliding portion 31 are rotated together, the outer casing 5 being mounted so as not to be rotatable to the rotation operation portion 32, and the inner cable 4 being mounted so as not to be rotatable to the sliding portion 31 will rotate together with the rotation operation portion 32 and the sliding portion 31. When the outer casing 5 and the inner cable 4 are rotated, the grasp portion 2 coupled to the outer casing 5 and the inner cable 4 is rotated on the distal end side of the forceps device 1. During this rotation operation, since the inner cable 4 is fixed without relative rotation to the sliding portion 31 and to the grasp portion 2 on both the distal end side and the proximal end side, friction due to the relative rotation does not occur, so that wear of the inner cable 4 does not occur.

Next, after operating the grasp portion 2 by the rotating operation, the sliding portion 31 is slid, and the inner cable 4 is operated to open the forceps pieces 21, 22 of the grasp portion 2 so as to grasp the affected area. When the forceps piece 21 and 22 open, the entire forceps device 1 is operated so that the affected area comes in between the forceps pieces 21 and 22, the sliding portion 31 is slid again in the opposite direction to close the forceps pieces 21 and 22, and grasp the affected area. In the forceps device 1 of the present invention, the practitioner is able to operate without changing hands from the starting of the rotation operation until the affected area is grasped. That is, the rotation operation portion 32 can be rotated, for example, by the right hand while holding the grip portion 6 with the left hand, and since the sliding portion 31 is provided on the rotation operation portion 32 within reach of one hand, it is possible to operate the sliding portion 31 as it is with the right hand. Furthermore, not only during the rotation operation but also during the sliding operation, the grip portion 6 is grasped on the side closer to the body of the patient, so it is possible to prevent swinging due to the sliding operation. Therefore, the operation of the forceps device 1 is very easy, and there is no burden on the patient.

When the affected area is grasped by the forceps pieces 21, 22, in order to carry out cauterization of the affected area, the high frequency power source is connected via a cord to the high frequency power source terminal 8, and then the affected area is subjected to cauterization by sending a high frequency current to the forceps pieces 21 and 22. In this case, by providing the high frequency power source terminal 8 on the grip portion 6, even when the rotational operation of the grasp portion 2 is carried out, the position of the high frequency power source terminal 8 will not change from its initial position. Therefore, since it is possible to maintain the high frequency power source terminal 8 in the desired position where it is easy for a practitioner to connect the cord, the practitioner can carry out the operation easily.

As described above, the forceps device 1 of the present invention has a good operability when the practitioner such as a doctor operates the forceps device 1, wear in the connection portion of the inner cable 4 provided in the forceps device 1 is not likely to occur, and there is no burden on the patient.

### EXPLANATION OF SYMBOLS

1 Forceps device
2 Grasp portion
21, 22 Forceps pieces
23 Axis of rotation
24 Housing of grasp portion
3 Operation portion
31 Sliding portion
31a, 31b Flange portion
32 Rotation operation portion
32a Body of rotation operation portion
32b Rotating portion
32c Guiding portion
32d Cavity
4 Inner cable
5 Outer casing
51 Stepped portion
6 Grip portion
6a Insertion hole
6b Protrusion
6c Insertion port
7 Resin-coated portion
71 Engaging portion
8 High frequency power source terminal
L link mechanism

1. A forceps device comprising:
a grasp portion equipped with forceps pieces for grasping a tissue in a body cavity, being able to be opened and closed;
an operation portion equipped with a sliding portion for opening and closing the forceps pieces of the grasp portion and a rotation operation portion for rotating the grasp portion;
an inner cable for transmitting an operating force from the operation portion to the grasp portion , provided between the grasp portion and the operation portion;
an outer casing surrounding the inner cable;
a grip portion to be grasped when operating the operation portion; and
a tubular resin-coated portion for housing the outer casing rotatably, extending toward a grasp portion side from the grip portion,
wherein a proximal end side of the inner cable is mounted to the sliding portion so as not to be rotatable, and a distal end side of the inner cable is connected to the grasp portion,
a proximal end side of the outer casing is mounted non-rotatably to the rotation operation portion, and a distal end side of the outer casing is engaged rotatably with respect to the resin-coated portion and immovably in an axial direction with respect to the resin-coated portion,
the sliding portion is mounted non-rotatably and slidably to the rotation operation portion,
the forceps pieces of the grasp portion are opened and closed by sliding the sliding portion,
the inner cable and the outer casing are rotated around an axis by rotating the rotation operation portion to rotate the grasp portion, and
the rotation operation portion is rotatably connected to the grip portion, and is provided at a proximal end side of the grip portion.

2. The forceps device according to claim 1, wherein a high frequency power supply terminal is connected to the inner cable or the outer casing, and the inner cable or the outer casing has an electric conductivity for enabling to supply high frequency current to the grasp portion from the high frequency power source terminal.

3. The forceps device according to claim 2, wherein the high frequency power source terminal is penetrated through the grasp portion to be connected to the outer casing.

4. The forceps device according to claim 2, wherein the high frequency power source terminal is provided at a rotation operation portion side, and is connected to the inner cable or the outer casing.

5. The forceps device according to claim 1, wherein the rotation operation portion comprises a guiding portion for guiding the sliding portion, and a rotating portion provided closer to the proximal end side than the guiding portion and operated to perform the rotation operation.
